(19) United States
(12) Patent Application Publication
(10) Pub. No.: US 2015/0190426 A1
(43) Pub. Date: Jul. 9, 2015

(54) METHOD OF INEXPENSIVE TRANSEPITHELIAL DIALYSIS WITH HOT WATER BATH AND SORBENTS

(71) Applicant: Joel Steven Goldberg, Hillsborough, NC (US)

(72) Inventor: Joel Steven Goldberg, Hillsborough, NC (US)

(21) Appl. No.: 14/211,899

(22) Filed: Mar. 14, 2014

Related U.S. Application Data
(60) Provisional application No. 61/923,818, filed on Jan. 6, 2014.

(51) Int. Cl. A61K 33/44

(52) U.S. Cl. CPC A61K 33/44

(57) ABSTRACT

Current technologies to treat end stage renal disease (ESRD) include peritoneal dialysis, hemodialysis and transplantation all of which are expensive. This invention is an inexpensive method to decrease body toxins that requires immersion of a body in a hot water bath with sorbents. In a hot water bath, secretory coils within activated sweat glands function as a semipermeable membrane that can dialyze toxins. Sorbents such charcoals and smectite clays located within the secretory coils of sweat glands in the vicinity of the zonula occludens can adsorb toxins. A subject who was immersed in a hot water bath with dissolved activated charcoal for a total time of approximately two hours presumably developed heparin deficiency from the adsorption of heparin onto activated charcoal. This invention is not expected to replace hemodialysis or peritoneal dialysis for the treatment of ESRD, but it may delay the initiation of traditional dialysis, decrease the number of high cost traditional dialysis procedures and improve quality of life. Patients who do not suffer from ESRD but who claim wellbeing from periodic detoxification may benefit from this invention. However, caution needs to exercised that unmonitored indiscriminate adsorption from hot water sorbent baths pose some dangers.

### CROSS-REFERENCES TO RELATED APPLICATIONS

This application claims the benefits of U.S. Provisional Patent Application No. 61/923,818 filed Jan. 6, 2014 each of which is incorporated herein by reference in its entirely.

### FEDERALLY FUNDED RESEARCH

Not applicable

### BACKGROUND OF THE INVENTION

As populations age, the incidence of end stage renal disease (ESRD) rises, causing increased expenditures for health care. Current technologies to treat ESRD include peritoneal dialysis, hemodialysis and transplantation, all of which are expensive. The costs of these technologies are not only the direct costs, but include treatment of complications including expensive medication and surgical therapies. Three direct causes of death from ESRD are hyperkalemia, toxemia and fluid overload. Hyperkalemia can cause cardiac asystole, toxins can cause encephalopathy and coma and fluid overload can cause congestive heart failure, pulmonary edema and hypoxemia. In this invention the activation of sweat coils within sweat glands provides a conduit with sufficient pore size such that the interstices between simple columnar epithelial cells of the coils function as a semipermeable membrane. Sorbents such as activated charcoal or smectite clays located in the vicinity of this semipermeable membrane can adsorb toxins from the plasma and detoxify the blood. This invention is an inexpensive method to reduce body toxins that may be used to delay the initiation of traditional dialysis, decrease the cost of traditional dialysis and provide a general method of detoxification.

Two major systems of kidney dialysis (hemodialysis and peritoneal dialysis) are in common use. Table 1 shows main features of hemodialysis as compared to this invention. These systems require electricity to monitor hydrostatics and power a pump, conduits, semipermeable membranes, expensive filters, dialysate and sorbents. In preparation for hemodialysis, patients need an arterial-venous vascular access. The simplicity of this invention compared to some methods of hemodialysis is showed in Table 1.

TABLE 1
Comparison of two hemodialysis systems to transepithelial dialysis

| Method | Electricity | Pump | Conduits | Membrane | Filters | Dialysate | Sorbents |
|---|---|---|---|---|---|---|---|
| Transepithelial | Optional to | − | − | − | − | − | + |
| dialysis | heat water | | | | | | |
| US | + | + | + | + | + | + | + |
| 20100004588 | | | | | | | |
| US | + | + | + | + | + | + | + or − |
| 20090127193 | | | | | | | |

Peritoneal dialysis requires less capital investment than hemodialysis but requires sterile dialysates and maintenance of an indwelling peritoneal catheter. Table 2 shows the main features of peritoneal dialysis compared to this invention.

TABLE 2
Comparison of peritoneal dialysis to transepithelial dialysis

| | Sterile | Intraperitoneal | Risk of | | | |
|---|---|---|---|---|---|---|
| | dialysate | catheter | infection | Bleeding | Pain | Sorbents |
| Transepithelial | No | No | Minimal | No | Minimal | Yes |
| dialysis | | | | | | |
| Peritoneal | Yes | Yes | Very | Low | High | No |
| dialysis | | | high | | | |

In search of prior art, a few reports were discovered supporting the complementary use of hot water or sauna baths to decrease serum concentrations of potassium and urea in patients with ESRD as proposed in this invention but none of these reports included the additional use of sorbents to improve the process. (Man in't Veld, van Maanen, & Schicht, 1978; Pruijm et al., 2013) Tables 3 and 4 summarize these observations.

TABLE 3
Urea and potassium losses in sweat and hemodialysis
in an anuric patient (Man in 't Veld, et al., 1978)

| | | | Sweat | | | Urea | Potassium |
|---|---|---|---|---|---|---|---|
| | Sweat: serum | Sweat: serum | rate | Urea | Potassium | losses/ | Losses/ |
| | urea | potassium | ml/min | losses | losses | week | week |
| Hot water | 1.8 | 2.5 | 33 | 2.6 g/hr | 12 meq/hr | 12.9 g | 60 meq |
| Sauna | 2.0 | 2.5 | 21 | | | | |
| Hemodialysis | | | | 7 g/hr | 20 meq/hr | 68 g | 200 meq |

TABLE 4
Potassium and urea loss in a pilot study of stimulated sweating
in hemodialysis patients (Pruijm, et al., 2013)

| | Control phase | Interventional period |
|---|---|---|
| Potassium (meq/L) | 5.1 | 5.0 |
| Urea (mmol/L) | 21.6 | 21.2 |

In prior art, sweating to eliminate body toxins depends upon the volume of sweat loss and the concentration of toxins dissolve in the sweat. Such systems can modestly complement the efficiency of hemodialysis. (Man in't Veld, et al., 1978) These systems can decrease intravascular volume and produce small changes in blood potassium and urea. Producing sufficient volume of sweat as a major method of detoxification, subjects the patient to risks of cardiovascular instability including increasing heart rate, decreasing blood pressure and hyperthermia. In this invention and in contrast to prior art, the volume of sweat loss is not the major process for detoxification. In this invention the activation of sweat coils of sweat glands provides a conduit with sufficient pore size such that the lateral basal membrane between simple columnar epithelial cells of the coils functions as a semipermeable membrane. Sorbents such as activated charcoal or smectite clays located in the vicinity of this semipermeable membrane can adsorb toxins from the plasma and detoxify the blood.

### DRAWINGS

FIG. 1 shows a cross section of a piece of skin. (Kenny) Label 1 shows the eccrine sweat coil, label 2 shows the apocrine sweat coil, label 3 shows a blood vessel and label 4 shows the exit of the sweat duct onto the skin. The duct size is sufficiently large to permit particles of charcoal and smectite clay to reside within the secretory coil and adsorb solutes from the transudate of the plasma.

FIG. 2 shows the physiology within the secretory coil according to the Na—K-2Cl model. (Saga, 2002) A transudate of the plasma exits through the lateral basal membrane and the solutes in the plasma are adsorbed by the sorbent labeled 1.

FIG. 3 is an electron micrograph that shows the anatomy of the lateral basal membrane. (Fawcett, 1966) Zonula occludens is labeled 1, zonula adherens is label 2, and desmosome is label 3. The lateral basal membrane forms a conduit through which transudate of the plasma from the blood vessel exits to the vicinity of the sorbent in the secretory coil.

### DETAILED DESCRIPTION OF THE INVENTION

Sweat gland activation produces a semipermeable membrane

“Certainly, the sweat glands could not be viewed as an efficient excretory organ that could satisfactorily substitute as a dialysis system for the elimination of metabolic end-products or in the maintenance of acid base equilibrium.” (page 71)(Ruth K Freinkel, 2001) The concepts and reduction to practice of this invention contradicts this statement of prior art.

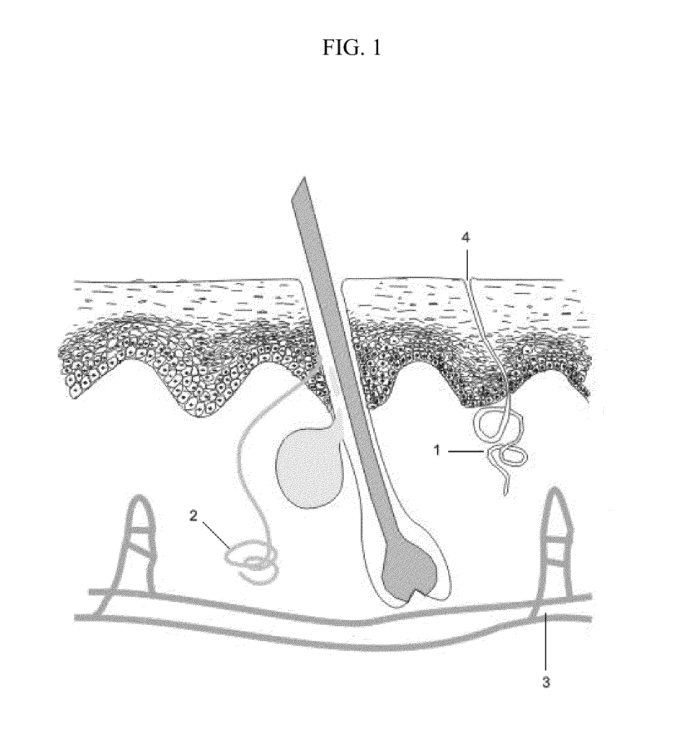

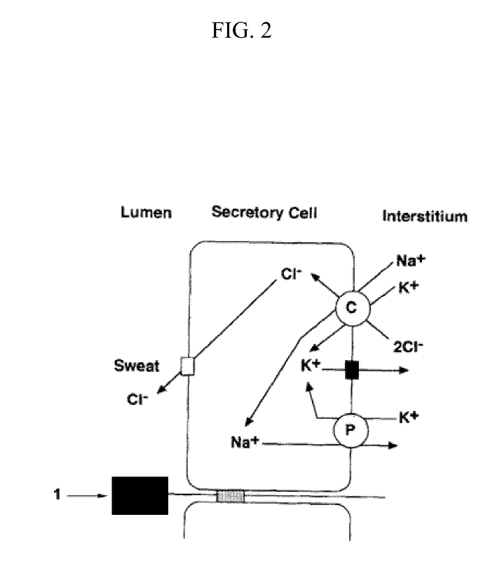

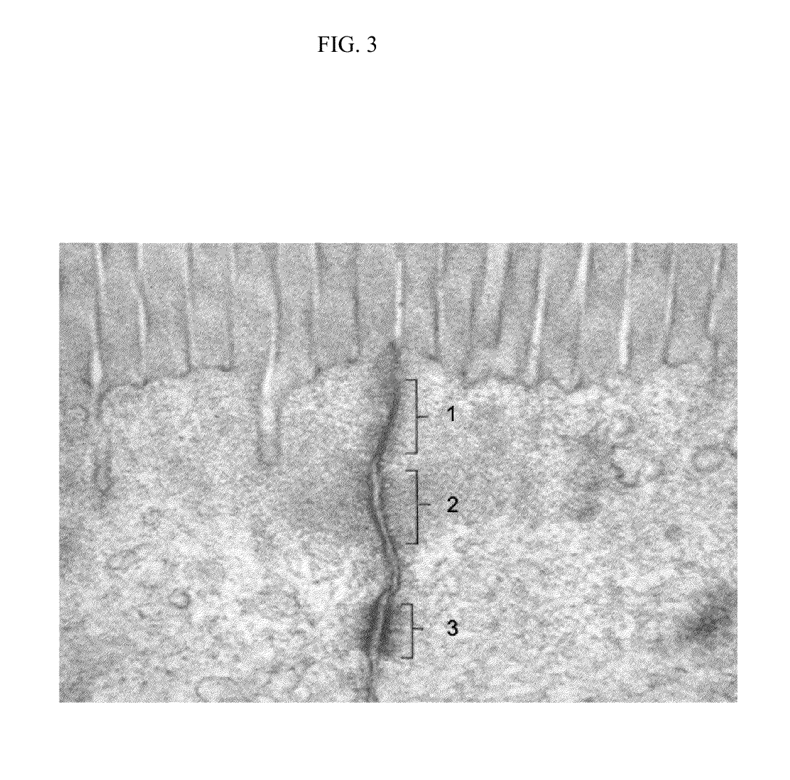

The two basic mechanisms that initiate sweating are: 1) hypothalamic signaling as a response to thermoregulation and 2) pharmacologic signaling predominantly through sympathetic afferent fibers. In both processes sweat is produced in the secretory coil, travels through the ducts and is expressed on the skin. (FIG. 1) The diameter of an apocrine secretory coil may be as large as 200 μm, versus the 60-80 μm diameter of an eccrine secretory coil. (Saga, 2002) Both coils are of sufficient size to permit sorbents to reside within the secretory coils with admixture of sweat and water bath. (FIG. 2) The most widely accepted mechanism for sweat production at the secretory coil is the Na—K-2Cl model in which secretion is coupled to transport across the lateral basal membrane where sweat comprised of plasma transudate can interface with a sorbent. (FIG. 2) Anatomically the lateral basal membrane corresponds to the zonula occludens, zonula adherens and desmosome that function as a semipermeable membrane with pore sizes of approximately 20 nm. (Riddle & Ernst, 1979) (FIG. 3) This pore size is large enough to permit transit of large molecules contained within the plasma into the secretory coil but small enough to block sorbent particles such as smectite clays with an average diameter of (1-2 μm) and activated charcoal powder with an average diameter of (1-150 μm) to enter the body. These relationships of secretory coil size, lateral basal membrane pore size and sorbent size permit adsorption to occur in the secretory coil without sorbent being introduced into the extra or intravascular fluids. Table 4 shows these relationships.

TABLE 4
Comparison of particle size of sorbents and pores sizes
of sweat secretory coils and lateral basal membrane
of columnar epithelium within the secretory coil

| | Pore or particle size | |
|---|---|---|
| Apocrine secretory coil | 200 | μm |
| Eccrine secretory coil | 60-80 | μm |
| Montmorillonite clay | 1-2 | μm |
| Bentonite clay | 1-2 | μm |
| Activated charcoal | 1-150 | μm |
| Zonula occludens | 20 | nm |
| Zonula adherens | 10-20 | nm |
| Desmosome | 30 | nm |

Ion exchange with smectite clays or adsorption with activated charcoal can detoxify substances at the interface of the basal lateral membranes of the secretory cells. The fluid that transfers through the membrane is a transudate of the plasma. Evidence to support the reduction to practice of this invention is provided in the experimental section that describes a subject immersed in a hot bath with dissolved activated charcoal for approximately two cumulative hours who developed a presumed heparin deficiency secondary to adsorption of heparin onto activated charcoal. The diagnosis of presumed heparin deficiency was based on the following abnormalities in the intrinsic clotting process: (details in experimental section)
1. The serum from the blood drawn prior to the experiment was liquid of normal consistency.
2. The serum from the blood drawn after the experiment was a dense gel.
3. The serum from the blood drawn that was mixed with heparin after the experiment was liquid of normal consistency
4. The serum from the blood drawn one week after the experiment was liquid of normal consistency as that of #1.
5. The serum from the blood drawn in a tube lightly coated with activated charcoal one week after the experiment was a dense gel and of the same consistency of the serum in #2.
6. At pH7.4 activated charcoal adsorbs very significant amounts of heparin. (Cooney, 1977)
7. Other anticoagulants such as antithrombin, protein s and protein c are not known to be adsorbed onto activated charcoal.
8. The time from blood draw to centrifugation was controlled.
9. Other causes to explain the observation including dehydration, premature centrifugation or temperature were excluded.
10. Coagulation factors are decreased when blood is hemoperfused over activated charcoal. (Winchester et al., 1978)

Characteristics of Smectite Clay as a Sorbent (Table 5)

Previous work has shown that some forms of clay can absorb potassium and urea. (Long, 2009; Muravyov, 2006; YEH, 2010) Smectite clays, specifically bentonite and more specifically montmorillonite contain layers of octahedral sheets sandwich between layers of tetrahedral sheets (TOT). Hydrated cations are located within the parallel clay layers. The cations of mainly Na, Mg, and Ca are only loosely held within the layers and can be exchanged for cations including those of potassium and urea. Water is attracted to the cations and results in swelling of the distance between the layers that further loosens the attraction permitting cation exchange. Previous work has also showed that smectite clays can adsorb urea although the chemistry of this process is not well understood, but it is likely that a cationic form of urea is exchanged. (Mortland, 1966) It is further predicted that other cations such as positively charge creatinine with an isoelectric point (pI) of 11.19 dissolved in plasma at ph 7.4 will ion exchange with smectite clays. However, depending upon the patient's blood chemistries the adsorptive properties of smectite clays can be modified as a preferred embodiment.

Characteristics of Charcoal as a Sorbent (Table 5)

It is well know that charcoal because of its microspore structure is an excellent sorbent of organic compounds. The micropore structure of various charcoals from hardwood trees, softwoods and coconut shell differ not only in their initial structure but also differ from methods of carbonization. Furthermore, activation of charcoal by chemical or steam changes the micropore structure by increasing the surface area available for adsorption and generally increasing the size of the micropores. Activation of charcoal is a proprietary process so it is expected that activated charcoals differ in their absorptive properties. Because of particle size, purity and micropore structure the preferred USP certified activated charcoal powder as used in the experimental section was food grade activated charcoal with the following characteristics: Surface area (m² g) 1700 min, Iodine number (mg/g) 1550 min, Ash 3%, and Heavy metals 0.005% max. However, depending upon the patient's blood chemistries the adsorptive properties of activated charcoal can be modified as the preferred embodiment. Table 5 shows the known and predicted adsorption characteristics of smectite clays and activated charcoal.

TABLE 5
Characteristic adsorption of sorbents

| | Potas- | | Creat- | Uric | Non-charged |
|---|---|---|---|---|---|
| | sium | Urea | inine | acid | organic compounds |
| Bentonite | + | + | Likely + | − | − |
| Montmo- | + | + | Likely + | − | − |
| rillonite | | | | | |
| Activated | − | − | − | − | + |
| charcoal | | | | | |

Hot Water Bath is the Preferred Environment

In a hot bath the sweat on the skin cannot evaporate so the normal physiology associated with evaporative cooling does not occur. Although the heat of conduction of water far exceeds that of air, (which explains why significant sweating does not occur when a body is immersed in water at temperatures of 80-90° F.) during sustained immersion at higher temperatures the core body temperature will rise. (Sherwood & Huber, 2010) The rise in core temperature will activate the sympathetic nervous system producing sweat and as a consequence produces a plasma transudate that flows across the zonula occludens into the secretory coil where it can interface with sorbents.

One feature of this invention is that the dialysis or detoxification procedure may need to be performed for short periods (15-30 minutes in 98-102° F. water) multiple times during the day until the effects are equilibrated with the rate of rise of the toxins and/or decrease in the concentration of toxins by complementary hemodialysis or peritoneal dialysis. During immersion in a hot water bath, it is quite common for a body to safely lose 250 ml of sweat. The set point temperature in the hypothalamus determines when sweating begins. The head temperature and the skin temperature contribute to the set point temperature and elevation of the skin temperature will lower the set point temperature. In a hot bath the skin temperature approximates the bath temperature. The initiation of sweating for each individual can be derived empirically with knowledge of bath and oral temperature.

Sweat volume can be monitored in two ways. Either the increase in concentration of a solute in sweat can be measured in the water bath or the volume of sweat can be measured as a function of decrease in body weight while the patient is immersed in the hot water bath. Sweat loss, duration of bath and water temperature can be plotted as nomograms to optimize the treatment schedule.

After replacement with fluid (in most cases drinking water), and return of temperature and cardiovascular parameters to baseline (usually less than 30 minutes) the procedure can be repeated multiple times unit the adsorption of the toxins achieves the desired decrease in toxin concentrations. Careful replacement of sweat with an intravenous infusion is an alternative method of rehydration.

Complications of Transepithelial Dialysis

This invention even in its simplest form has some risks. Evidence that supports the reduction to practice of this invention shows that presumed heparin deficiency secondary to heparin adsorption by activated charcoal dissolved in a hot water bath can occur. Charcoal can indiscriminately adsorb organic molecules and smectite clays can exchange various cations. The cation exchange could introduce various metallic cations into the circulation. Hyponatremia, hypokalemia, hypomagnesemia and hypocalcemia are fluid exchange electrolyte problems that could also occur. Activation of sweat glands is associated with hemodynamic and thermal changes that could be deleterious. Finally, this invention may increase the incidence of infection in patients who maintain a peritoneal dialysis catheter.

Benefits to Society

Decrease Cost of Traditional Dialysis Treatment

Treatment for ESRD is very expensive with estimated 2012 costs to the US health care system of 85 billion dollars. Without development of a low cost technologic improvement, these costs are unlikely to decrease as the incidence of ESRD increases. Although this invention is not expected to replace existing technologies, it may delay the initiation of traditional dialysis, decrease the required number of traditional dialysis procedures and improve quality of life. The technology can eliminate body toxins without ingestion or colonic administration of sorbents commonly used to manage hyperkalemia in patients with ESRD. This invention may also improve overall wellbeing in patients who do not suffer from ESRD but use sorbents for detoxification.

Decrease Human Exposure to Toxins

In the last 150 years, a myriad of new chemical compounds have been synthesized to improve man's standard of living. Examples include food additives, agricultural fertilizers and pesticides, pharmaceuticals and plastics, just to name a few. (Crinnion, 2000a, 2000b, 2000c, 2000d, 2010) In the history of man's evolution, these are new substances, unknown to our ancestors that can accumulate in the body to levels in which they become toxins. The liver, kidney and intestines are major body organs to detoxify these chemicals, but the capacities of these organs may be exceeded. The deleterious effects from these toxins may present as diseases such as chronic fatigue syndrome, gulf war syndrome or environmental hypersensitivity syndrome or more insidiously as perceptional disturbances of the brain to include some forms of mental illness. The brain, the most protected organ within the human body, has activity described as chaotic rather than stochastic or periodic. (Faure & Korn, 2001; Sarbadhikari & Chakrabarty, 2001; Wang, Meng, Tan, & Zou, 2010) It is well established that small perturbations in a chaotic system can have profound effects on the trajectory of that system. Thus, exposure of unfamiliar chemicals to the nervous system at a sufficient dose over time may lead to disturbances. The fetus, through placental transfer, may also be exceptionally sensitive to unabated chemical exposure.

It is unlikely that the rate of newly synthesized chemicals will decrease, on the contrary, advances in technology favor synthesizing more substances. Hot water bathing, practiced for centuries, has been supplanted with showers in many societies and heavy labor associated with prolific sweating has decreased as work has become more mechanize. If the adsorptive process described in this invention can be safely controlled, as a general method of detoxification, this invention may have uses beyond improving care for those suffering ESRD.

Experimental Data

Table 7 shows changes in core temperature, blood pressure, heart rate and weight associated with fluid loss during activation of sweat glands in a body immersed in a hot tub. Weight loss in the form of sweat can be regulated by the time and percent of body immersed in the hot water and the temperature of the water bath. The water bath of highest temperature did not produce the greatest weight loss. This can occur because the hypothalamic response to hyperthermia is also regulated by the body's response to dehydration and hypernatremia. Recovery of core body temperature, heart rate and blood pressure is rapid post-immersion.

TABLE 7
Pre and post immersion of a body in a hot water bath

| | Water | | | | |
|---|---|---|---|---|---|
| Time | temp- | Core | Blood | | Weight |
| (minutes) | erature ° F. | temperature ° F. | pressure | Pulse | (lbs.) |
| Trial #1 | | | | | |
| 0 | 102 | 98.6 | 137/87 | 77 | 222 |
| (Pre- | | | | | |
| immersion) | | | | | |
| 30 | 101 | 100.5 | 122/66 | 127 | 220 |
| (Post- | | | | | |
| immersion) | | | | | |
| 35 | | 98.7 | 140/75 | 96 | |
| 40 | | 98.0 | 129/74 | 93 | |
| 45 | | 98 | 124/82 | 91 | |
| 75 | | 98 | 133/75 | 76 | |
| Trial #2 | | | | | |
| 0 | 100 | 96 | 158/81 | 82 | 220 |
| (Pre- | | | | | |
| immersion | | | | | |
| 30 | 99.5 | 99.5 | 130/77 | 132 | 219.5 |
| (Post | | | | | |
| immersion) | | | | | |
| 35 | | 99 | 135/89 | 100 | |
| 40 | | 99 | 137/88 | 100 | |
| 45 | | 98.8 | 142/87 | 94 | |
| 75 | | 98.2 | 143/89 | 90 | |
| Trial #3 | | | | | |
| 0 | 103.5 | 97.5 | 132/85 | 88 | 223 |
| (Pre- | | | | | |
| immersion) | | | | | |
| 15 | 103.0 | 100.8 | 117/74 | 130 | 222 |
| (Post- | | | | | |
| immersion) | | | | | |
| 20 | | 98.6 | 120/80 | 113 | |
| 25 | | 98.4 | 121/83 | 94 | |
| 30 | | 98.6 | 130/90 | 90 | |

Table 8 shows the changes in vital signs and intake and output of fluids during immersion of a subject in a hot water bath with dissolved activated charcoal and bentonite. Total immersion time was 105 minutes over a 9.7 hour period.

TABLE 8
Immersion in hot water bath with sorbents

| | | | Temp | Wt. | Charcoal | Bentonite | Urine | Fluid | Water temp |
|---|---|---|---|---|---|---|---|---|---|
| Time | BP | Pulse | ° F. | lbs | grams | grams | ml | ml | ° F. |
| 0 | 136/82 | 66 | 96 | 222 | 3.5 | 8.5 | | | 103 |
| 15 | 94/65 | 128 | 99.8 | 221.5 | | | | | 102 |
| 60 | 128/74 | 80 | 97.8 | 222 | 3.5 | | | 240 | 102 |
| 75 | 93/65 | 138 | 99.7 | 220 | | | 300 | | 101 |
| 120 | 119/76 | 97 | 97.6 | 222.5 | 3.5 | | | 720 | 103 |
| 135 | 84/62 | 136 | 99.8 | 222.5 | | | | | 103 |
| 180 | 123/90 | 107 | 97.8 | 223 | 3.5 | | | 720 | 103 |
| 195 | 111/75 | 138 | 99.0 | 223.5 | | | | | |
| 540 | 140/82 | 96 | 97.3 | 224 | 3.5 | 8.5 | 1000 | | 102 |
| 555 | 103/70 | 140 | 98.9 | 223.5 | | | | | 102 |
| 570 | 124/85 | 89 | 99.2 | 223.5 | | | | | 102 |
| 585 | 104/68 | 124 | 99.5 | 222 | | | 400 | | 102 |

In the experiment shown in Table 8 there were no differences in the pre and post immersion measurements of serum electrolytes (Na, K, Cl) or blood urea nitrogen (BUN) or creatinine (Cr). This was not totally unexpected because the primary sorbent, activated charcoal, does not avidly adsorb these chemistries. However unexpectedly, the finding that serum in the post immersion sample had formed a dense gel led to a presumed diagnosis of heparin deficiency as described below.

Presumed experimentally produced heparin deficiency from immersion in hot water bath with dissolved activated charcoal based on blood analysis

A sample of 10 ml of pre-immersion blood was obtained in a plastic syringe through a 19 G needle, transferred to a tube without additives and the sample was centrifuged at 2700 rpm for 10 minutes. The serum was poured into a second tube and frozen to be assayed at a later date.

Twelve hours after the immersion experiment as shown in Table 8 had concluded a 10 ml blood sample was obtained in a plastic syringe through a 19 G needle, transferred to a tube without additives and the sample was centrifuged at 2700 rpm for 10 minutes. The serum consisted of a dense gel and the test tube could be inverted without loss or movement of contents. A second sample was drawn and centrifuged under identical conditions and again the serum consisted of a dense gel and the test tube could be inverted without loss or movement of contents. A third sample was obtained in a lithium heparin coated tube centrifuged under identical conditions and the serum had normal consistency and was frozen for assay.

One week after the immersion experiment blood samples were obtained in a plastic syringe through a 19 G needle, transferred to a tube without additives and the sample was centrifuged at 2700 for 10 minutes. The serum was liquid of the same consistency as in the pre-immersion specimen. A second sample was collected in a tube without additive coated with activated charcoal centrifuged at 2700 for 10 minutes. The serum was a dense gel with the same consistency of the post-immersion sample.

The diagnosis of presumed heparin deficiency was made on the basis of these observations, exclusion of other possibilities and the known significant adsorption of heparin on non-coated activated charcoal. (Cooney, 1977) Fibrin formation has been reported in blood collected in a non-additive tube and prematurely centrifuged but all the above samples were centrifuged within the range of 2-3 minutes after phlebotomy and the dense gel of serum was of the same consistency as that of the activated charcoal treated tube. This observation supports the reduction to practice of this invention. Therefore in a human, activated charcoal dissolved in a hot water bath can presumably adsorb heparin. This process is extended to adsorption of toxins or ion exchange by other sorbents such as smectite clays. The adsorption takes place in the secretory coils of the sweat glands by the mechanism described in this invention. It was fortunate that the subject did not suffer a thromboembolic event as a consequence of this experiment.

### REFERENCES

Cooney, D. O. (1977). Heparin adsorption on activated charcoal. Clin Toxicol, 11(5), 569-572.
Crinnion, W. J. (2000a). Environmental medicine, part 2—health effects of and protection from ubiquitous airborne solvent exposure. Altern Med Rev, 5(2), 133-143.
Crinnion, W. J. (2000b). Environmental medicine, part 4: pesticides—biologically persistent and ubiquitous toxins. Altern Med Rev, 5(5), 432-447.
Crinnion, W. J. (2000c). Environmental medicine, part one: the human burden of environmental toxins and their common health effects. Altern Med Rev, 5(1), 52-63.
Crinnion, W. J. (2000d). Environmental medicine, part three: long-term effects of chronic low-dose mercury exposure. Altern Med Rev, 5(3), 209-223.
Crinnion, W. J. (2010). The CDC fourth national report on human exposure to environmental chemicals: what it tells us about our toxic burden and how it assist environmental medicine physicians. Altern Med Rev, 15(2), 101-109.
Faure, P., & Korn, H. (2001). Is there chaos in the brain? I. Concepts of nonlinear dynamics and methods of investigation. C R Acad Sci III, 324(9), 773-793.
Fawcett, D. (1966). The Cell: An Atlas of Fine Structure. Philadelphia: WB Saunders. Kenny, Beverley. With permission to modify.
Long, L., Zhang, Y, Wang, X., Cao, Y. (2009). Montmorillonite adsorbs urea and accelerates urea excretion from the intestine. Applied Clay Science, 46(1), 57-62.
Man in't Veld, A. J., van Maanen, J. H., & Schicht, I. M. (1978). Stimulated sweating in chronic renal failure. Br Med J, 2(6131), 172-173.
Mortland, M. M. (1966). Urea complexes with montmorillonite: An infrared absorption study. Clay Minerals, 6, 143-156.
Muravyov, V. I., Sakharov, B. A. (2006). Experimental study of the sorption of potassium by montmorillonite. Sedimentology, 15(1-2), 103-112.
Pruijm, M., El-Housseini, Y., Mahfoudh, H., Jarraya, F., Hachicha, J., Teta, D., et al. (2013). Stimulated sweating as a therapy to reduce interdialytic weight gain and improve potassium balance in chronic hemodialysis patients: a pilot study. Hemodial Int, 17(2), 240-248.
Riddle, C. V., & Ernst, S. A. (1979). Structural simplicity of the zonula occludens in the electrolyte secreting epithelium of the avian salt gland. J Membr Biol, 45(1-2), 21-35.
Ruth K Freinkel, D. T. W. (Ed.). (2001). The Biology of the Skin. Carnforth: Parthenon Publishing.
Saga, K. (2002). Structure and function of human sweat glands studied with histochemistry and cytochemistry. Prog Histochem Cytochem, 37(4), 323-386.
Sarbadhikari, S. N., & Chakrabarty, K. (2001). Chaos in the brain: a short review alluding to epilepsy, depression, exercise and lateralization. Med Eng Phys, 23(7), 445-455.
Sherwood, S. C., & Huber, M. (2010). An adaptability limit to climate change due to heat stress. Proc Natl Acad Sci USA, 107(21), 9552-9555.
Wang, X., Meng, J., Tan, G., & Zou, L. (2010). Research on the relation of EEG signal chaos characteristics with high-level intelligence activity of human brain. Nonlinear Biomed Phys, 4(1), 2.
Winchester, J. F., MacKay, J. M., Forbes, C. D., Courtney, J. M., Gilchrist, T., & Prentice, C. R. (1978). Hemostatic changes induced in vitro by hemoperfusion over activated charcoal. Artif Organs, 2(3), 293-300.
YEH, R. H. e. a. (2010). U.S. Patent No. 20100004588

Having described my invention, I claim:

1. A method to reduce body toxins by immersing a body and inducing sweat in a hot water bath comprised of sorbents.

2. The method of claim 1 that includes toxins comprising potassium, urea, creatinine, organic or non-organic compounds or of any mixture of these.

3. The method of claim 1 that includes use of sorbents such as those from smectite clays, ion exchange resins, charcoal, activated charcoal or biochar or any mixtures of these.

4. The method of claim 1 with multiple hot water baths.
